(19) United States
(12) Patent Application Publication
(10) Pub. No.: US 2002/0150560 A1
(43) Pub. Date: Oct. 17, 2002

(54) Treatment of Il-10 deficiencies

(76) Inventors: Daniel Sauder, Toronto (CA); Arkady Mandel, North York (CA); Anthony Bolton, Derbyshire (GB)

Correspondence Address: BURNS DOANE SWECKER & MATHIS L L P, POST OFFICE BOX 1404, ALEXANDRIA, VA, 22313-1404, US

(21) Appl. No.: 10/030,418

(22) PCT Filed: May 11, 2001
(86) PCT No.: PCT/CA01/00679

(30) Foreign Application Priority Data
May 11, 2000 (CA) 2,308,105

(51) Int. Cl. A61K 45/00; A61K 33/00

(52) U.S. Cl. 424/93.7; 604/506; 424/600

(57) ABSTRACT

Mammalian disorders associated with a deficiency in the cytokine interleukin-10 (IL-10) can be alleviated by stimulating in vivo secretion and/or activity of IL-10 in the patient's blood or tissues by application of external stimulus.

### FIELD OF THE INVENTION

This invention relates to the cytokine inteleukin-10 (hereafter IL10), and methods for the treatment or prophylaxis of mammalian disorders associated with IL-10 deficiency.

### BACKGROUND OF THE INVENTION AND PRIOR ART

It is known that IL-10, originally described as cytokine synthesis inhibitory factor, plays a role in suppressing immune and inflammatory responses in the mammalian body, by inhibiting the production of pro-inflammatory cytokines. A deficiency of IL-10 results in the development of a number of significant inflammatory events including ischemia-reperfusion injury, and has been implicated in autoimmune diseases such as psoriasis and pemphigus. It has been reported to be a Th2-derived cytokine that inhibits the cytokine release by Th1 cells (see Biorencino et al., J. Exp. Med. 170: 3081-2095, 1989). Studies of the biologic activities of IL-10 in vitro have shown that IL-10 inhibits production of cytokines at both mRNA and protein levels by mouse Th1 clones stimulated by antigen or CD3 antibody in the presence of macrophages (see, again, the above cited paper by Biorencino et al.).

Kondo. McKenzie and Sauder, “The Journal of Investigative Dermatology,” Vol. 103, 1994, pages 811-814 have reported that IL-10 application suppresses interferon gamma mRNA up-regulation in challenged skin, suggesting that IL-10 significantly modifies the elucidation of allergic contact sensitivity reactions.

The administration of exogenous IL-10 as a therapeutic agent to treat IL-10 deficiency-associated disorders in a mammalian patient, on any significant scale, is currently unattractive. The preparation of IL-10 by chemical synthesis, or by cell cultivation and expression techniques (e.g., using recombinant DNA technologies) is prohibitively expensive.

Current treatment of pemphigus is with oral steroids, usually prednisone. Immunosuppressive drugs such as Imuran and Cytoxan are also used as adjunctive therapy to the steroid treatment. However, these drugs have serious side effects and, accordingly, treatment for pemphigus which has reduced side effects is needed.

### SUMMARY OF THE INVENTION

It has now been found that increased secretion of IL-10 can be caused in vivo, in a mammalian patient, and that such enhanced secretion of IL-10 in vivo has a beneficial, therapeutic effect on a wide variety of mammalian disorders, including, but not limited to inflammatory disorders and disorders arising from a deficient immune system or a deficient endothelial function in the patient.

One aspect of the present invention is a method for the prophylactic or therapeutic treatment of inflammatory components and inflammatory aspects of an IL-10 deficiency-mediated disease in a mammalian patient, which method comprises administering to said patient an aliquot of blood which has been treated ex vivo with at least one stressor selected from the group consisting of an oxidative environment, thermal stress and UV light, wherein the concentration of IL-10 secreted by immune cells in the blood or tissues of said patient is increased, with an associated reduction of harmful inflammatory effects of the IL-10 deficiency-mediated disease.

In another embodiment, the method comprises (a) identifying a patient having an IL-10 deficiency-mediated disease condition, or is at risk of having an IL-10 deficiency-mediated disease condition, which has a significant inflammatory component; (b) evaluating the patient identified in (a) above to determine whether that disease condition or risk of disease condition can be effectively treated by increasing the concentration of IL-10 in the patient; and (c) if an increase in the concentration of IL-10 would be suitable for the prophylactic or therapeutic treatment of such a disease, then administering to said patient an aliquot of blood which has been treated ex vivo with at least one stressor selected from the group consisting of an oxidative environment, thermal stress and UV light.

Another aspect of the invention is a method for preventing or treating an IL-10 deficiency-associated disorder in a mammalian patient which method comprises (a) identifying a patient having an IL-10 deficiency-mediated disorder which disorder is characterized by a decreased amount of IL-10 secretion; (b) evaluating the patient to determine whether that disorder can be effectively treated by increasing the amount of IL-10 secretion; and (c) administering to the patient an effective amount of extracorporeally stressed blood to stimulate IL-10 secretion in the patient.

Yet another aspect of this invention is a method for preventing or treating an IL-10 deficiency-mediated disorder in a mammalian patient, which method comprises (a) selecting a patient having an IL-10 deficiency-mediated disorder which disorder is characterized by a decreased amount of IL-10 secretion; (b) evaluating the patient to determine whether that disorder can be effectively treated by stimulating IL-10 secreting cells; and (c) administering to the patient an effective amount of extracorporeally stressed blood to stimulate IL-10 secreting cells thereby increasing the relative proportion of IL-10 secreting cells in the mammalian patient and stimulating the secretion of IL-10.

Another aspect of the invention is a method for preventing or treating an IL-10 deficiency-mediated disorder in a mammalian patient, which method comprises (a) selecting a patient having an IL-10 deficiency-mediated disorder which disorder is characterized by a decreased amount of IL-10 activity; (b) evaluating the patient to determine whether that disorder can be effectively treated by increasing the activity of IL-10 in the patient; and (c) administering to the patient an effective amount of extracorporeally stressed blood to increase IL-10 activity in the patient.

Another aspect of the invention provides a process of increasing in vivo levels of IL-10 in a mammalian patient, by increasing the number or relative proportion of IL-10-secreting cells in the mammalian body or by increasing the amount of IL-10 secretion from IL-10-secreting cells in the mammalian body, or by increasing the activity of IL-10 secreted in the mammalian body, and thereby effecting beneficial therapeutic effects in the patient.

From another aspect, the invention provides biologically acceptable compositions of matter administrable to a mammalian patient, and which, upon or after such administration, stimulate enhanced secretion of IL-10 in vivo in the mammalian patient, for treatment or prophylaxis of various mammalian disorders.

### BRIEF DESCRIPTION OF THE DRAWINGS

The figures of accompanying drawings are graphical presentations of results obtained according to specific examples described below.

FIG. 1 shows a comparison of ear thickness measurement in mice following treatment as described in Example 1.

FIG. 2 is a cross section of the ear of animals treated as described in Example 1.

FIG. 3 shows a comparison of net ear swelling in mice following treatment as described in Example 2.

FIG. 4 shows a comparison of net ear swelling in mice following treatment as described in Example 3.

FIG. 5 shows a comparison of net ear swelling in mice following treatment as described in Example 4.

### DETAILED DESCRIPTION OF THE INVENTION

This invention is directed to methods for the treatment or prophylaxis of mammalian disorders associated with IL-10 deficiency, in particular disease such as pemphigus and psoriasis. The following terms are defined with respect to this application.

“Pemphigus” is a group of autoimmune diseases characterized by the formation of watery blisters on the skin. It is an intraepidermal blistering disease characterized clinically by superficial blisters and erosions of the skin and/or mucous membranes, especially the mouth. It is currently treated with massive doses of steroids.

It is classified into three major forms: “pemphigus foliaceus,” a superficial and more benign form; “pemphigus vulgaris,” a deeper, more serious and more common form; and “paraneoplastic pemphigus,” the rarest and most serious form which occurs most often in patients who have been diagnosed with a malignancy (cancer).

The forms have minor variants. Pemphigus foliaceus (“PF”) is caused by the location of the blister just below the stratum corneum of the epidermis. Oral lesions are uncommon. Pemphigus vulgaris (“PV”) patients generally present in their fifth to sixth decade, exhibiting blisters and erosions on skin and mucous surfaces. Oral lesions often precede skin lesions by a few months. The lesions appear as erosions due to the fragility of the blisters. The oral lesions can spread to the pharynx and the larynx, with resulting hoarseness, excruciating pain, and inability to eat and drink. It has a grave prognosis if not treated. Before the introduction of oral steroid treatments, mortality was 100% by 5 years, due to malnutrition, debilitation, sepsis and/or electrolyte and water loss. Paraneoplastic pemphigus patients generally exhibit sores in the mouth, lips and esophagus and skin lesions of different types occur. This form of the disease is usually unresponsive to standard steroid treatment. In some cases, surgical removal of the associated tumor can lead to improvement of the symptoms of pemphigus. Cotell, S.; Robinson, N. D.; and Chan, L. S., American Journal of Emergency Medicine, (May 2000) Vol. 18, No.3, page 1, further describe the various types of pemphigus.

Toto, P., Feliciani C, Amerio P, Suzuki H, Wang B, Shivji G M, Woodley D and Sauder D N, J. Immunol (Jan 1, 2000) 164(1):522-9, report that pemphigus vulgaris (“PV”) is characterized by antibodies to the desmosomal cadherin desmoglein-3. This work, on delineating the potential role of T-cell signalling pathways in the pathogenesis of PV, involved passive transfer experiments with PV IgG in gene targeted mutant mice. Data described therein suggests a potential role of IL-10 in PV.

The term “prophylaxis” refers to the prevention of disease or preventive treatment of a disease.

Patients having IL-10 deficiency-associated disorders can be identified by an examination of their symptoms. The presence of the visible symptoms of psoriasis or pemphigus on a patient's skin, or the presence of symptoms of inflammatory bowel disease in a patient, are evidence of IL-10 deficiency. Such identification can also be made, or confirmed, by measurement of IL-10 and IL-10 mRNA in affected tissues of the patient, in the patient's blood and/or the patient's blood cells. IL-10 measurements can be conducted by methods well known to those skilled in the art. See, for example, the aforementioned papers of Biorencino et al. and Kondo et al., page 1 hereof, and especially references cited therein. Commonly, antibody measurements are used, in plasma or tissue, as with measurements of other cytokines.

A patient thus identified as having an IL-10 deficiency-associated disorder may be evaluated to determine whether that disorder can be effectively treated by increasing the amount of in vivo IL-10 secretion, by administering IL10 from an external source and observing the resultant effect on the patient's symptoms of this disorder. IL-10 is commercially available, at least in test quantities. It can be administered to the test patient intravenously or by local injection at the site of the disorder, as a liquid infusion in a biologically acceptable carrier.

One way to enhance IL-10 secretion is by introduction into the patient of compatible whole blood which has been extracorporeally subjected to an oxidative stress. Another is by introduction into the patient of compatible whole blood which has been subjected extracorporeally to an appropriate dose of UV radiation. Yet another is by introduction into the patient of compatible Whole blood which has been subjected extracorporeally to an oxidative stress and an appropriate dose of UV radiation simultaneously.

Still other processes are the introduction into the patient of a cellular fraction of compatible blood, optionally including platelets, which has been subjected extracorporeally to oxidative stress, or one which has been subjected to an appropriate dose of UV radiation extracorporeally, or one which has been subjected extracorporeally to both an oxidative stress and an appropriate dose of UV radiation simultaneously. In all cases it is preferred to use the patient's own blood or blood cells, for extracorporeal processing and introduction into the patient.

A preferred process of the present invention involves extraction of an aliquot of blood from a mammalian subject (preferably a human), and treatment of the aliquot of blood or a separated cellular fraction thereof ex vivo, simultaneously or sequentially, with the oxidative stress and UV radiation. Then it is injected back into the same subject. Preferably a combination of both of the oxidative stressor and the UV radiation is used simultaneously. The result is an enhanced secretion of IL-10 in the mammalian patient.

Preferably also, the aliquot of blood is in addition subjected to mechanical stress. Such mechanical stress is suitably that applied to the aliquot of blood by extraction of the blood aliquot through a conventional blood extraction needle, or a substantially equivalent mechanical stress, applied shortly before the other chosen stressors are applied to the blood aliquot. This mechanical stress may be supplemented by the mechanical stress exerted on the blood aliquot by bubbling gases through it, such as ozone/oxygen mixtures, as described below. Optionally also, a temperature stressor may be applied to the blood aliquot, simultaneously or sequentially with the other stressors, i.e., a temperature at, above or below body temperature.

The terms “aliquot,” “aliquot of blood” or similar terms used herein include whole blood, separated cellular fractions of the blood including platelets, separated non-cellular fractions of the blood including plasma, plasma components and combinations thereof. Preferably, in human patients, the volume of the aliquot is up to about 400 ml, preferably from about 0.1 to about 100 ml, more preferably from about 1 to about 15 ml, even more preferably from about 8 to about 12 ml, and mot preferably about 10 ml. When a cellular fraction is used instead of whole blood, the aliquot should contain the number of blood cells which would ordinarily be contained in whole blood of the aforementioned volumes, e.g., 10³-10¹². The effect of the combination of stressors is to modify the blood, and/or the cellular or non-cellular fractions thereof, contained in the aliquot. The modified aliquot is then re-introduced into the subject's body by any suitable method, most preferably intramuscular injection, but also including subcutaneous injection, intraperitoneal injection, intra-arterial injection, intravenous injection and oral administration.

The optionally applied temperature stressor either warms the aliquot being treated to a temperature above normal body temperature or cools the aliquot below normal body temperature. The temperature is selected so that the temperature stressor does not cause excessive hemolysis in the blood contained in the aliquot and so that, when the treated aliquot is injected into a subject, the desired effect will be achieved. Preferably, the temperature stressor is applied to that the temperature of all or a part of the aliquot is up to about 55° C., and more preferably in the range of from about −5° C. to about 55° C.

In some preferred embodiments of the invention, the temperature of the aliquot is raised above normal body temperature, such that the mean temperature of the aliquot does not exceed a temperature of about 55° C., more preferably from about 40° C. to about 50° C., even more preferably from about 40° C. to about 44° C., and most preferably about 42.5±1° C.

In other preferred embodiments, the aliquot is cooled below normal body temperature such that the mean temperature of the aliquot is within the range of from about 4° C. to about 36.5° C., more preferably from about 10° C. to about 30° C., and even more preferably from about 15° C. to about 25° C.

The oxidative stressor can be the application to the aliquot of solid, liquid or gaseous oxidizing agents. Chemical oxidants such as hydrogen peroxide can be used. Preferably, it involves exposing the aliquot to a mixture of medical grade oxygen and ozone gas, most preferably by applying to the aliquot medical grade oxygen gas having ozone as a component therein. The ozone content of the gas stream and the flow rate of the gas stream are preferably selected such that the amount of ozone introduced to the blood aliquot, either on its own or in combination with one of the other stressors, does not give rise to excessive levels of cell damage, and so that, when the treated aliquot is injected into a subject, the desired effect will be achieved. Suitably, the gas stream has an ozone content of up to about 300 μg/ml, preferably up to about 100 μg/ml, more preferably about 30 μg/ml, even more preferably up to about 20 μg/ml, particularly preferably from about 10 μg/ml to about 20 μg/ml, and most preferably about 14.5±1.0 μg/ml. The gas stream is suitably supplied to the aliquot at a rate of up to about 2.0 liters/min, even more preferably up to about 0.33 liters/min, and most preferably about 0.24±0.024 liters/min. The lower limit of the flow rate of the gas stream is preferably not lower than 0.01 liters/min, more preferably not lower than 0.1 liters/min, and even more preferably not lower than 0.2 liters/min.

The ultraviolet light, which may also be regarded as a stressor, is suitably applied by irradiating the aliquot under treatment from a source of UV light. Preferred UV sources are UV lamps emitting UV-C band wavelengths, i.e. at wavelengths shorter than about 280 nm. Ultraviolet light corresponding to standard UV-A (wavelengths from about 315 to about 400 nm) and UV-B (wavelengths from about 280 to about 315) sources can also be used. As in the case of the oxidative stressor, the UV dose should be selected, on its own or in combination of the other chosen stressor(s), so that excessive amounts of cell damage do not occur, and so that, when the treated aliquot is injected into a subject, the desired effect will be achieved. For example, an appropriate dosage of such UV light, can be obtained from up to eight lamps arranged to be exposed to the sample container holding the aliquot, operated at an intensity to deliver a total UV light energy at 253.7 nm at the surface of the blood of from about 0,025 to about 10 joules/cm², preferably from about 0.1 to about 3.0 joules/cm². Such a treatment, applied in combination with the oxidative environment stressor, provides a modified blood aliquot which is ready for injection into the subject, to cause enhanced levels of IL-10 to be generated in vivo in the subject.

It is preferred to subject the aliquot to the oxidative environment stressor, the UV light stressor and the temperature stressor simultaneously, following the subjection of the aliquot to the mechanical stress, e.g. by extraction of the blood from the patient. Thus, the aliquot may be maintained at a predetermined temperature above or below body temperature while the oxygen/ozone gas mixture is applied thereto and while it is irradiated with ultraviolet light.

The time for which the aliquot is subjected to the stressors is normally within the time range of from about 0.5 minutes up to about 60 minutes. The time depends to some extent upon the chosen combination of stressors. When UV light is used, the intensity of the UV light may affect the preferred time. The chosen temperature level may also affect the preferred time. When oxidative environment in the form of a gaseous mixture of oxygen and ozone applied to the aliquot is chosen as one of the two stressors, the concentration of the oxidizing agent and the rate at which it is supplied to the aliquot may affect the preferred temperature. Some experimentation to establish optimum times may be necessary on the part of the operator, once the other stressor levels have been set. Under most stressor conditions, preferred times will be in the approximate range of from about 2 to about 5 minutes, more preferably about 3 minutes. The starting blood temperature, and the rate at which it can be warmed or cooled to a predetermined temperature, tends to vary from subject to subject. Warming is suitably by use of one or more infrared lamps placed adjacent to the aliquot container. Other methods of warming can also be adopted.

As noted, it is preferred to subject the aliquot of blood to a mechanical stressor, as well as the chosen stressor(s) discussed above. Extraction of the blood aliquot from the patient through an injection needle constitutes the most convenient way of obtaining the aliquot for further exitracorporeal treatment, and this extraction procedure imparts a suitable mechanical stress to the blood aliquot. The mechanical stress may be supplemented by subsequent processing, for example the additional mechanical shear stress caused by bubbling as the oxidative stressor is applied.

In the practice of the preferred process of the present invention, the blood aliquot may be treated with the heat, UV light and oxidative environment stressors using an apparatus of the type described in aforementioned U.S. Pat. No. 4,968,483 to Mueller. The aliquot is placed in a suitable, sterile container, which is fitted into the machine. A UV-permeable container is sued and the UV lamps are switched on for a fixed period before the other stressor is applied, to allow the output of the UV lamps to stabilize. When a temperature stressor is used combination, the UV lamps are typically on while the temperature of the aliquot is adjusted to the predetermined value, e.g. 42.5±1° C. Four UV lamps are suitably used, placed around the container.

In the administration of the extracorporeally treated aliquot to the patient for the stimulation of IL-10 secretion in the patient's body, a mammalian patient is preferably given one or more courses of treatments, each course of treatment comprising the administration to a mammalian subject of one or more (e.g. one to six) aliquots of mammalian blood modified as discussed above.

The pharmaceutical compositions of this invention may optionally include a pharmaceutically acceptable excipient. Some examples of suitable excipients include sterile water, sterile saline, phosphate buffered saline, and the like.

For optimum effectiveness of the treatment, it is preferred that no more than one aliquot of modified blood be administered to the subject per day, in one or more injection sites, and that the maximum rest period between any two consecutive aliquots during the course of treatment be no greater than about 21 days. Booster treatments as described below may advantageously be used. As used herein, the term “rest period” is defined as the number of days between consecutive aliquots or consecutive courses of treatment on which no aliquots of modified blood are administered to the subject.

Therefore, except where aliquots are administered to the subject on consecutive days, a rest period of from 1 to 21 days is provided between any two aliquots during the course of treatment. Moreover, at least one of the rest periods during the course of treatment preferably has a length of about 3 to 15 days.

Although it may be sufficient to administer only one course of treatment as described above to the subject, it may be preferred in some circumstances to administer more than one course of treatment, or to follow the above-described course of treatment by periodic “booster” treatments, if necessary, to maintain the desired effects of the present invention. For example, it may be preferred to administer booster treatments at intervals of 3 to 4 months following the initial course of treatment, or to administer a second course of treatments to the subject following a rest period of several weeks or months.

The process of the present invention increases the IL-10 level in a mammalian patient's body, both in tissue and in blood, and accordingly shows potential in the treatment and prophylaxis of a wide variety of inflammatory events and other disorders which are associated with IL-10 deficiencies.

The invention is further illustrated and described with reference to the following specific examples, comprising animal studies conducted in an approved manner. The system chosen to demonstrate the role and effect of enhanced secretion of IL-10 in mammalian patients is contact hypersensitivity, a T-cell mediated delayed-type hypersensitivity reaction in which the skin of a patient exhibits a reaction to an agent which the body has previously encountered, by contact or by inoculation. There is available an established experimental mouse model for induction and testing treatments of this disorder, as described in the following Examples. In addition genetically modified laboratory mice, specifically bred to produce no IL-10, are available commercially to permit testing of the effects of various processes on production, see Example 4 below.

Patients having IL-10 deficiency-associated disorders can be identified by an examination of their symptoms. The presence of the visible symptoms of psoriasis or pemphigus on a patient's skin, or the presence of symptoms of inflammatory bowel disease in a patient, are evidence of IL-10 deficiency. Such identification can also be made, or confirmed, by measurement of IL-10 and IL-10 mRNA in affected tissues of the patient, in the patient's blood and/or the patient's blood cells. IL-10 measurements can be conducted by methods well known to those skilled in the art—see for example the aforementioned papers of Biorencino et al. and Kondo et al., page 1 hereof, and especially references cited therein. Commonly, antibody measurements are used, in plasma or tissue, as with measurements of other cytokines.

A patient thus identified as having an IL-10 deficiency-associated disorder may be evaluated to determine whether that disorder can be effectively treated by increasing the amount of in vivo IL-10 secretion, by administering IL10from an external source and observing the resultant effect on the patient's symptoms of this disorder. IL-10 is commercially available, at least in test quantities. It can be administered to the test patient intravenously or by local injection at the site of the disorder, as a liquid infusion in a biologically acceptable carrier.

### EXAMPLE 1

The effectiveness of the treatment according to a preferred embodiment of the present invention, on contact hypersensitivity (CHS), was assessed on laboratory mice, according to approved animal experimentation procedures, using the method described by Kondo et al., “Lymphocyte function associated antigen-1 (LFA-1) is required for maximum elicitation of allergic contact dermatitis” Br J. Dermatol. 131:354-359, 1994, with minor variations. The disclosure thereof is incorporated herein by reference. Briefly, to induce CHS, the abdominal skin of each mouse was shaved and painted with dinitrodifluorobenzene DNFB, the sensitizing chemical, using 25 μl of 0.5% DNFB in 4:1 acetone: olive oil solution. This sensitization was applied to four groups of five Balb C mice.

Whole blood was obtained from Balb/c mice, by extraction from a main artery through an injection needle, and treated with an anti-coagulant. An aliquot of this was subjected to the process of a preferred embodiment of the invention, to obtain treated blood. The remainder was left untreated, for use in control experiments. Since these mice are genetically identical, the administration of the treated blood to others of the group is equivalent to administration of the treated blood to the donor animal.

To obtain treated blood, the selected aliquot, in a sterile, UV-transmissive container, was treated simultaneously with a gaseous oxygen/ozone mixture and ultraviolet light at elevated temperature using an apparatus as generally described in aforementioned U.S. Pat. No. 4,968,483 Mueller et al. Specifically, 10 ml of citrated blood was transferred to a sterile, low density polyethylene vessel (more specifically, a Vasogen VC7002 Blood Container) for ex vivo treatment with stressors according to the invention. Using an apparatus as described in the aforementioned Mueller patent (more specifically, a Vasogen VC7001 apparatus), the blood was heated to 42.5±1° C. and at that temperature irradiated with UV light principally at a wavelength of 253.7 nm, while oxygen/ozone gas mixture was bubbled through the blood to provide the oxidative environment and to facilitate exposure of the blood to UV. The constitution of the gas mixture was 14.5±1.0 μg ozone/ml, with the remainder of the mixture comprising medical grade oxygen. The gas mixture was bubbled through the aliquot at a rate of 240±24 ml/min for a period of 3 minutes.

Of the 4 groups of sensitized mice, the first, control group A-1 received no treatment. The second, control group B-1, was treated with physiological saline, 50 μl. The third, control group C-1, was sham treated, with 50 μl of blood which had been extracted but not treated with the stressors. The fourth, test group D-1, was treated with 50 μl of blood subjected to stressors as described above. Treatments, each involving intramuscular injection of 50 μl of the respective liquid, started on the day of sensitization, and was repeated every day for a total of 6 days. On the same day as the last treatment, but after its; administration, the animals were challenged with DNFB, by applying to the ears of each animal 10 μl of 0.2% solution of DNFB. Inflammation due to CHS manifests itself in a swelling of the ears. Ear thickness was measured, 24 hours after challenge, with a Peacock spring-loaded micrometer (Ozaki Co., Tokyo, Japan). The results were expressed as the change (from pre-challenge level) in ear thickness and represent the mean maximal increase at 24 hours after challenge.

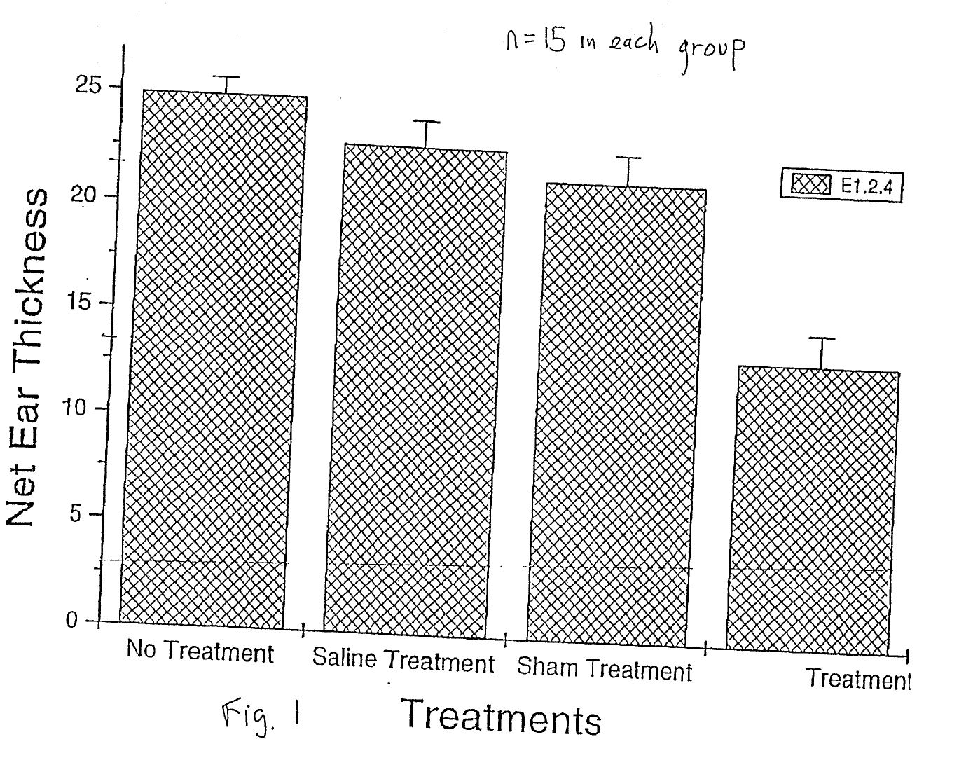

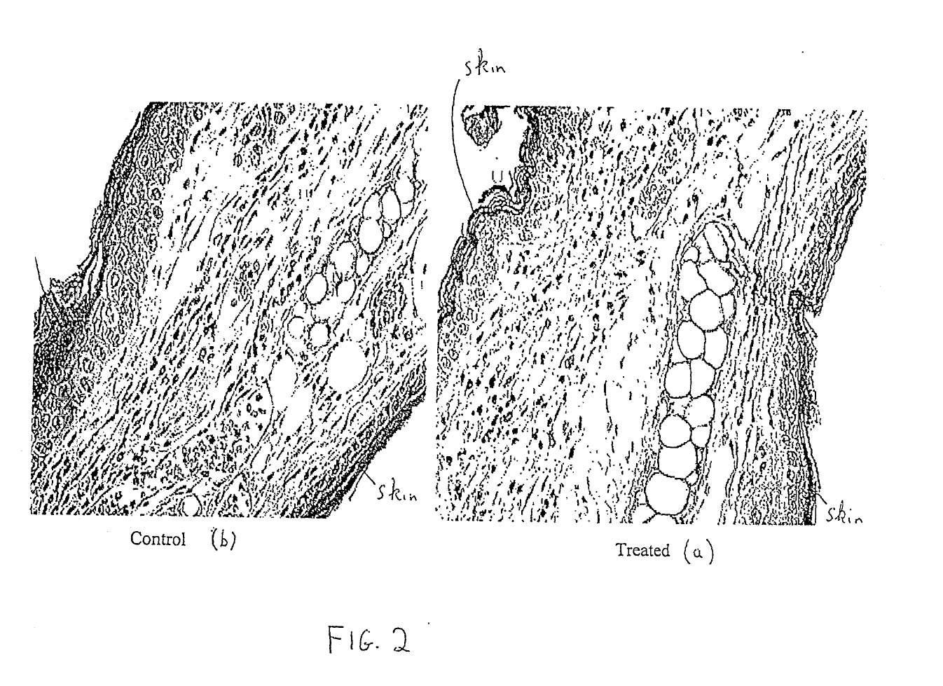

The experiments were repeated two more times, using two more sets of four groups of animals, to ensure statistical significance in the results. FIG. 1 of the accompanying drawings is a graphical presentation of these results. A notable and significant reduction in ear thickness (inflammation) is to be observed with the animals treated according to this preferred process of the invention, as compared with any of the other groups. FIG. 2 of the accompanying drawings represent photographs of cross-sections of the ears of a representative treated animal of group D-1 (picture (a)) and a representative untreated group A-1 animal (picture (b)). The decreased skin thickness, and the reduced lymphocyte infiltration (lower density of dark stained cells) is readily apparent on picture (a) from the treated animal, further demonstrating a significant reduction in inflammation.

The percentage suppression when compared with the standard CHS response (no treatment, control group A-1) is 8% for the saline treatment group B-1, 14% for the sham treatment group C-1 and 46% for group D-1, treated according to the embodiment of the process of the invention.

### EXAMPLE 2

The procedure of Example 1 was followed, using four groups of Balb/c mice, with one group receiving a blood aliquot which had been subjected to UV and ozone/oxygen bubbling, as described, but without application of the heat stressor (i.e. treated at room temperature). Thus, group A-2 received no treatment, group B-2 received untreated blood (sham treatment), group C-2 received blood treated with UV and ozone but no heat, and group D-2 received blood treated the same way as in the case of group D-1 of Example 1.

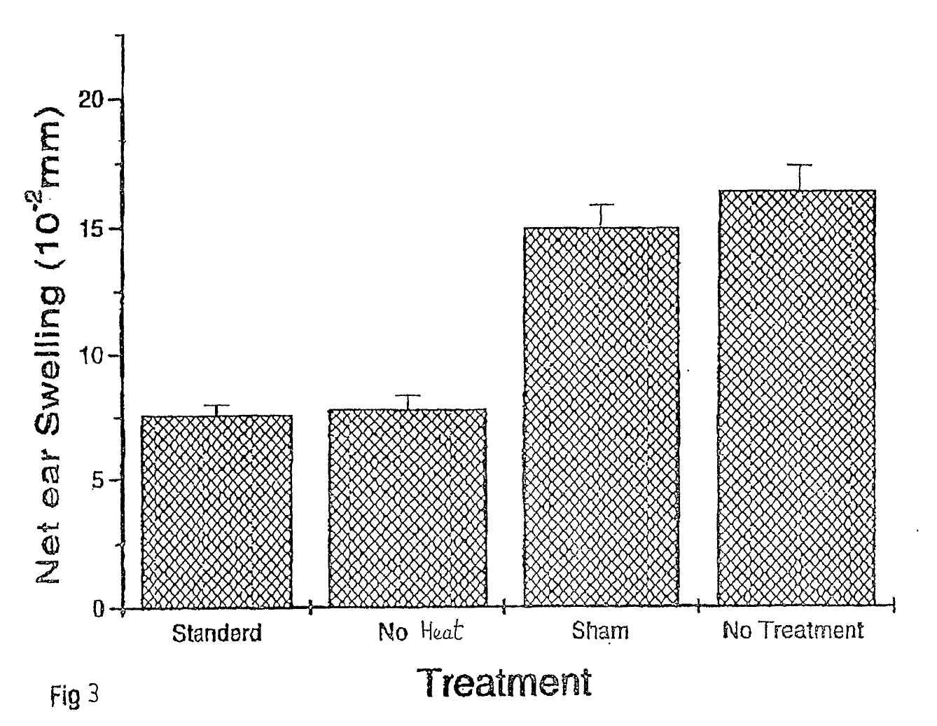

The results are presented graphically on FIG. 3, in the same manner as

FIG. 1. The result from group D-2 is marginally better than that from group C-2. The percentage suppression when compared to the standard CHS response (no treatment, group A-2) is 9% for group B-2, sham treatment, 52.5% for group C-2 and 54% for group D-2.

### EXAMPLE 3

Whole blood was obtained from Balb/c mice. Part of the blood was subjected to UV, ozone and heat treatment as described in Example 1, and part of the blood remained untreated. Both the untreated blood and the treated blood were centrifuged to obtain a cellular fraction, and washed with saline. The treated and untreated fractions were administered to animals challenged with DNFB to develop contact hypersensitivity as described in Example 1.

Four groups of 5 mice each were injected according to the schedule of Example 1, and evaluated, as follows: Group A-3—no-treatment; Group B-3—cellular fraction of sham treated blood; Group C-3—cellular part of treated blood; Group D-3—whole treated blood. The administrations to the mice took place just prior to sensitization with 0.5% DNFB and continued every day until challenge with 0.2% DNFB, 5 days later. A total of 6 injections were administered to each mouse.

The ear swelling of each mouse was measured 24 hours after challenge. Each experiment was repeated three times, to ensure statistical significance of the results. Net ear swelling as a measure of contact hypersensitivity and suppression thereof was calculated as 1—(ear swelling of blood administer mouse/ear swelling of no blood administered mouse)×100.

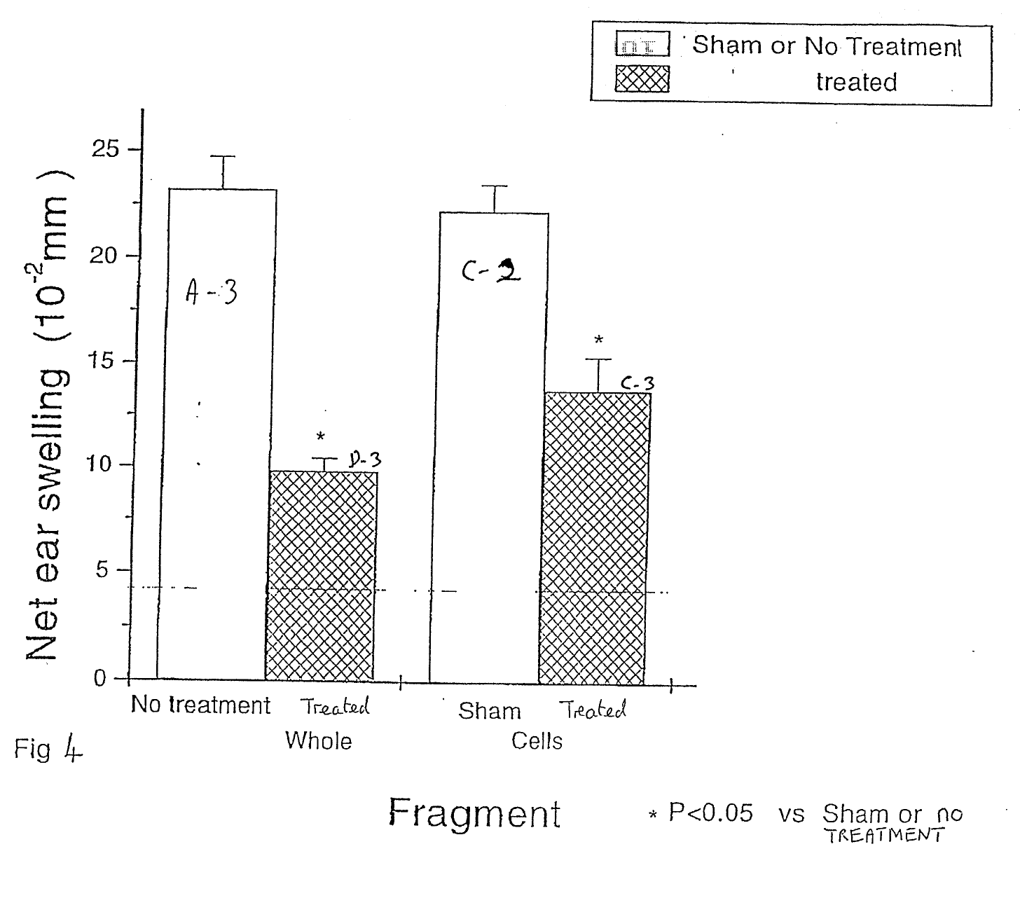

The results are presented graphically on FIG. 4, a summary of three experiments. A significant suppression of CHS is seen with the cellular fraction of the treated blood. There was no significant difference between the treated cellular fraction and treated whole blood.

### EXAMPLE 4

To demonstrate the fundamental role of IL-10 secretion in the processes described above, the procedure of Example 1 was essential repeated, using a genetic strain of laboratory mice deficient in the gene responsible for IL-10 production and secretion, i.e. IL-10 knock-out mice. These are available from laboratory animal sources, for approval experimental purposes.

Four groups each comprising five IL-10 knock-out mice were sensitized with DNFB, as described in Example 1. Whole blood was obtained from the IL-10 knock-out mice, by extraction from a main artery through an injection needle, and treated with an anti-coagulant. Aliquots of this blood were treated as described in Example 1, and other aliquots left-untreated for use as controls.

Control group A-4 received no injection. The animals of Control group B-4 were treated with physiological saline. The animals of control Group C-4 were then treated with 50 μl of blood which had been extracted but not treated with stressors. The fourth test group of animals D-4 were treated with 50 μl of blood subjected to stressors as described. The treatment schedules, challenge with DNFB and measurement of results via ear swelling were all as described in Example 1.

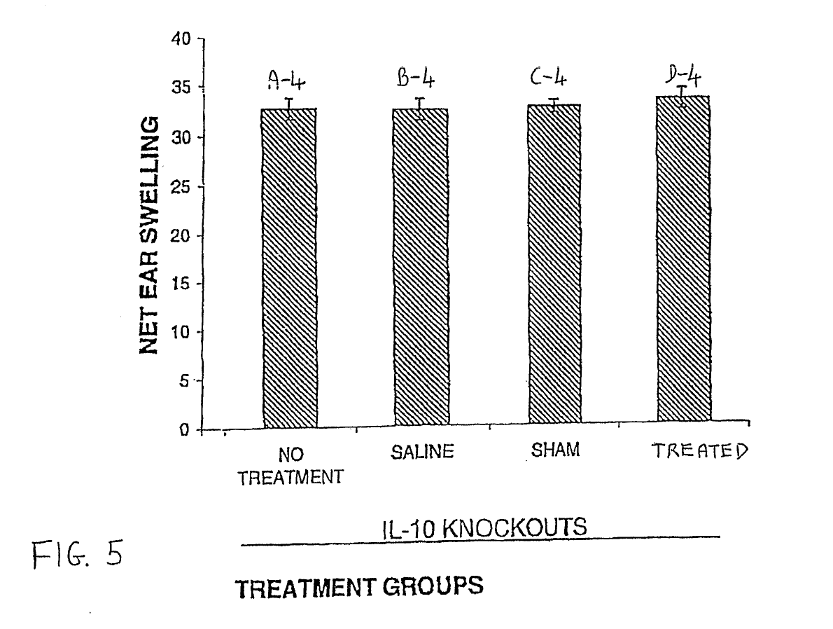

The results were shown graphically on accompanying FIG. 5. There is no difference between any of the four groups. This demonstrates that the treatment according to the invention is ineffective when applied to IL-10 knock-out mice whereas it is very effective when applied to mice of the same genetic background but expressing the IL-10 gene, so that IL-10 secretion is a key function in the treatment.

What is claimed is:

1. A method for the prophylaxis or therapeutic treatment of an IL-10 deficiency-mediated disease in a mammalian patient, which method comprises:
administering to said patient an aliquot of blood which has been treated ex vivo with at least one stressor selected from the group consisting of an oxidative environment, thermal stress and UV light, wherein the concentration of IL-10 secreted by immune cells in the blood or tissues of said patient is increased, with an associated reduction of harmful inflammatory effects of the IL-10 deficiency-mediated disease.

2. The method of claim 1, wherein the oxidative environment comprises applying an oxidizing agent to the aliquot.

3. The method of claim 2, wherein the oxidative agent comprises ozone gas, and the ozone gas is introduced into the blood aliquot in ah amount which does not give rise to excessive levels of cell damage.

4. The method of claim 2, wherein the oxidizing agent comprises a mixture of ozone gas and medical grade oxygen, the ozone gas being contained in the mixture in a concentration of up to about 300 μg/ml.

5. The method of claim 4, wherein the ozone gas is contained in the mixture in a concentration of up to about 30 μg/ml.

6. The method of claim 5, wherein the ozone gas is contained in the mixture in a concentration of from about 13.5 μg/ml to about 15.5 μg/ml.

7. The method of claim 4, wherein the mixture is applied to the aliquot at a flow rate of up to about 0.33 liters/min.

8. The method of claim 7, wherein the mixture is applied to the aliquot at a flow rate of from about 0.21 liters/min to about 0.27 liters/min.

9. The method of claim 1, wherein the electromagnetic emission comprises ultraviolet light having one or more UV-C band wavelengths.

10. The method of claim 1, wherein the temperature to which the aliquot is cooled or heated is a temperature which does not result in substantial hemolysis of the blood in the aliquot.

11. The method of claim 1, wherein the temperature stressor is applied so that the temperature of at least part of the aliquot is in the range of from about −5° C. to about 55° C.

12. The method of claim 1, wherein the mean temperature of the blood in the aliquot is in the range of from about 37° C. to about 44° C.

13. The method of claim 1, wherein the mean temperature of the blood in the aliquot is in the range of from about 0° C. to about 36.5° C.

14. The method of claim 1, wherein the mean temperature of the blood in the aliquot is in the range of from about 10° C. to about 30° C.

15. The method of claim 1, wherein the temperature is in the range of from about 37° C. to about 55° C.

16. The method of claim 15, wherein the temperature is 42.5±1° C.

17. The method of claim 1, wherein the volume of the aliquot is up to about 400 ml.

18. The method of claim 17, wherein the volume of the aliquot is about 10 ml.

19. The method of claim 17, wherein the volume of the aliquot is about 2 ml.

20. The method of claim 1, wherein the aliquot is subjected to the stressors for a period of up to about 60 minutes.

21. The method of claim 20, wherein the aliquot is subjected to the stressors for a period of about 3 minutes.

22. The method of claim 1, wherein the blood is administered to the mammal by a method suitable for vaccination selected from the group consisting of intra-arterial injection, intramuscular injection, intravenous injection, subcutaneous injection, intraperitoneal injection, and oral, nasal or rectal administration.

23. The method of claim 1, wherein all of the stressors are simultaneously administered to the aliquot.

24. The method of claim 1, wherein any two of the stressors are simultaneously administered to the aliquot.

25. The method of claim 24 wherein the mammal is a human.

26. The method of claim 25 wherein the IL-10 deficiency-mediated disease is pemphigus.

27. The method of claim 1 including the additional steps of:
(a) identifying a patient having an IL-10 deficiency-mediated disease condition, or is at risk of having an IL-10 deficiency-mediated disease condition, which has a significant inflammatory component;
(b) evaluating the patient identified in (a) above to determine whether that disease condition or risk of disease condition can be effectively treated by increasing the concentration of IL-10 in the patient; and
(c) if an increase in the concentration of IL-10 would be suitable for the prophylactic or therapeutic treatment of such a disease, then administering to said patient an aliquot of blood which has been treated ex vivo with at least one stressor selected from the group consisting of an oxidative environment, thermal stress and UV light.

28. A method for the prophylactic or therapeutic treatment of inflammatory components and inflammatory aspects of an IL-10 deficiency-mediated disease in a mammalian patient, which method comprises:
(a) identifying a patient having an IL-10 deficiency-mediated disease condition, or is at risk of having an IL-10 deficiency-mediated disease condition, which has a significant inflammatory component;
(b) evaluating the patient identified in (a) above to determine whether that disease condition or risk of disease condition can be effectively treated by increasing the concentration of IL-10 in the patient; and
(c) if an increase in the concentration of IL-10 would be suitable for the prophylactic or therapeutic treatment of such a disease, then administering to said patient an aliquot of blood which has been treated ex vivo with at least one stressor selected from the group consisting of an oxidative environment, thermal stress and UV light;
wherein the concentration of IL-10 secreted by immune cells in the blood or tissues of said patient is increased, with an associated reduction of harmful inflammatory effects of the IL-10 deficiency-mediated disease.

29. A method for preventing or treating an IL-10 deficiency-associated disorder in a mammalian patient which method comprises (a) identifying a patient having an IL-10 deficiency-mediated disorder which disorder is characterized by a decreased amount of IL-10 secretion; (b) evaluating the patient to determine whether that disorder can be effectively treated by increasing the amount of IL-10 secretion; and (c) administering to the patient an effective amount of extracorporeally stressed blood to stimulate IL-10 secretion in the patient.

30. A method for preventing or treating an IL-10 deficiency-mediated disorder in a mammalian patient, which method comprises (a) selecting a patient having an IL-10 deficiency-mediated disorder which disorder is characterized by a decreased amount of IL-10 secretion; (b) evaluating the patient to determine whether that disorder can be effectively treated by stimulating IL-10 secreting cells; and (c) administering to the patient an effective amount of extracorporeally stressed blood to stimulate IL-10 secreting cells thereby increasing the relative proportion of IL-10-secreting cells in the mammalian patient and stimulating the secretion of IL-10.

31. A method for preventing or treating an IL-10 deficiency-mediated disorder in a mammalian patient, which method comprises (a) selecting a patient having an IL-10 deficiency-mediated disorder which disorder is characterized by a decreased amount of IL-10 activity; (b) evaluating the patient to determine whether that disorder can be effectively treated by increasing the activity of IL-10 in the patient; and (c) administering to the patient an effective amount of extracorporeally stressed blood to increase IL-10 activity in the patient.

32. The method according to claim 29, 30 or 31 wherein the extracorporeally stressed blood is compatible whole blood or blood cells.

33. The process of claim 32 wherein the extracorporeal stress is an oxidative stress.

34. The process of claim 32 wherein the extracorporeal stress is UV radiation.

35. The process of claim 32 wherein the extracorporeal stress comprises simultaneously applying oxidative stress and UV radiation.

36. A method for increasing IL-10 levels in the blood and/or tissues of a mammalian patient, which method comprises extracting an aliquot of the patient's blood, extracorporeally applying to at least the cellular portion of the aliquot at least one stressor selected from UV radiation and oxidative stress, and re-administering the resultant aliquot to the mammalian patient.

37. The method of claim 36 wherein both the UV radiation stressor and the oxidative stressor are extracorporeally applied to the aliquot simultaneously.

38. The process of claim 36 or 37 wherein the oxidative stress is bubbling of a mixture of medical grade oxygen and ozone through the aliquot.

39. A pharmaceutical composition for administration to a mammalian patient comprising patient-compatible IL-10 secretion-stimulating mammalian blood cells wherein the blood cells stimulate an increase in IL-10 levels in the blood and/or tissues of the mammalian patient when administered to the mammalian patient.

40. The pharmaceutical composition of claim 39 wherein the blood cells are extracorporeally stressed with at least stressor selected from the group consisting of oxidative stress and ultraviolet radiation stress.

41. A process of alleviating the symptoms of, or prophylaxes of, an IL-10 deficiency-associated disorder in mammalian patient, which comprises in vivo stimulation of enhanced IL-10 secretions in the mammalian patient by application to the patient of IL-10 secretions stimulating extracorporeally stressed compatible whole blood or blood cells.

42. A process of alleviating the symptoms of, or prophylaxes of, an IL-10 deficiency-associated disease in a mammalian patient, which comprises in vivo increasing the number of relative proportion of IL-10-secreting cells in the mammalian patient and stimulating IL-10 secretion therefrom, by application to the patient of IL-10 cell enhancing extracorporeally stressed compatible whole blood or blood cells.

43. A process of alleviating the symptoms of, or prophylaxes of, an IL-10 deficiency-associated disorder in a mammalian patient, which comprises in vivo enhancing the activity of IL-1-in the mammalian patient's body by application to the patient of IL-10 activity increasing extracorporeally stressed compatible whole blood or blood cells.

44. The process of claim 41, 42 or 43 wherein the extracorporeal stress is an oxidative stress.

45. The process of claim 41, 42 or 43 wherein the extracorporeal stress is UV radiation.

46. The process of claim 41, 42 or 43 wherein the extracorporeal stress is a combination of oxidative stress and UV radiation, applied extracorporeally to the whole blood or cellular fraction thereof simultaneously.

47. A process of increasing IL-10 levels in the blood and/or tissues of a mammalian patient, which comprises extracting an aliquot of the patient's blood, extracorporeally applying to at least the cellular portion of the aliquot at least one stressor selected from UV radiation and oxidative stress, and re-administering the resultant aliquot to the mammalian patient.

48. The process of claim 48 wherein both the UV radiation stressor and the oxidative stressor are extracorporeally applied to the aliquot simultaneously.

49. The process of claim 47 or claim 48 wherein the oxidative stress is bubbling of a mixture of medical grade oxygen and ozone through the aliquot.

50. A biologically acceptable composition of matter for administration to a mammalian patient, said composition of matter comprising extracorporeally stressed compatible mammalian blood cells which have been subjected to at least one of oxidative stress and ultraviolet radiation stress, said composition of matter having the ability, upon administration to the mammalian patient, of stimulating an increase in IL-10 levels in the blood and/or tissues of the mammalian patient.
